(19) United States
(12) Patent Application Publication
(10) Pub. No.: US 2005/0234402 A1
(43) Pub. Date: Oct. 20, 2005

(54) Fluid dispensing device

(76) Inventors: Matthew Spencer Collins, Henlow (GB); Michael Birsha Davies, Ware (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/523,256

(22) PCT Filed: Jul. 30, 2003
(86) PCT No.: PCT/EP03/08497

§ 371 Date: Jan. 27, 2005

(30) Foreign Application Priority Data
Aug. 1, 2002 (GB) 0217802.8

(51) Int. Cl. A61M 1/00

(52) U.S. Cl. 604/151

(57) ABSTRACT

A fluid dispensing device having a housing and a pump action fluid discharge device. The pump action fluid discharge device is arranged to be actuated by one or more levers which are pivotally connected to part of the housing and are driveably connect by a toothed drive to a container forming part of the fluid discharge device 8. When the one or more levers are squeezed together the container is urged towards a nozzle of the fluid dispensing device causing a pump forming part of the fluid discharge device to dispense a single dose of fluid from the nozzle.

The present invention relates to a medicament dispenser and in particular to a fluid dispensing device for use as a nasal inhaler.

It is well known to provide a medicament dispenser in which fluid is dispensed via a nozzle or orifice upon the application of a force by a user to a single actuation lever or button. Such devices may be arranged to dispense a single dose or may alternatively be arranged with a reservoir containing several doses to be dispensed.

The Applicants have now found that for ease of use and efficiency of dispensing of fluid (e.g. as a spray) it is advantageous if the lever is provided to the housing of a medicament dispenser device such that it is pivotally supported within the housing but driveably connects with a fluid container within the housing to enable the container to be urged towards a dispensing position. Ease of use benefits can arise because a so-configured dispenser may be arranged to be ergonomically amenable to the user. Efficiency benefits can arise because such an arrangement of the lever can provide good mechanical advantage even for a relatively compact dispenser device housing.

It is an object of this invention to provide a fluid dispensing device that is easier to use and in particular a device which provides a more efficient dispensing of fluid.

According to a first aspect of the invention there is provided a fluid dispensing device for spraying a fluid into a body cavity comprising a housing, a nozzle for insertion into a body cavity, a fluid discharge device moveably housed within the housing, the fluid discharge device comprising a container for storing the fluid to be dispensed and a compression pump having a suction inlet located within the container and a discharge outlet at one end of the container for transferring fluid from the pump to the nozzle and a finger operable means to apply a force to the container to move the container towards the nozzle so as to actuate the pump wherein the finger operable means comprises of at least one lever pivotally supported within the housing and driveably connected to the container so as to urge the container towards the nozzle when the or each lever is actuated by a user.

Suitably, the or each lever is arranged to apply mechanical advantage. That is to say, the or each lever applies mechanical advantage to the user force to adjust (generally, to enhance or smooth) the force experienced by the container. The mechanical advantage may in one aspect, be provided in either a uniform manner such as by a constant mechanical advantage enhancement, for example by a ratio of from 1.5:1 to 10:1 (enhanced force: initial force), more typically from 2:1 to 5:1. In another aspect, the mechanical advantage is applied in a non-constant manner such as progressive increase or progressive decrease of mechanical advantage over the applied force cycle. The exact profile of mechanical advantage variation may be readily determined by reference to the desired spray profile and all relevant characteristics of the device and formulation to be sprayed (e.g. viscosity and density).

In one aspect, there are two opposing levers each of which may be pivotally connected to part of the housing and may be driveably connected to the container so as to urge the container towards the nozzle when the levers are squeezed together by a user.

The or each lever may be driveably connected to the container near to said one end of the container.

The or each lever may have a toothed portion for engagement with a toothed rack attached to the container so as to form the driveable connection therebetween.

The container may have a longitudinal axis and the or each toothed rack may extend parallel to the longitudinal axis of the container.

Each toothed rack may have two sets of opposed teeth, a first set of teeth for engagement with a first lever and a second set of teeth for engagement with a second lever.

The container may have a neck portion at said one end and the or each toothed rack may be attached to the neck portion of the container so as to form in combination with the or each lever the driveable connection.

Suitably, the container may have two toothed racks attached thereto.

The two toothed racks may be arranged on opposite sides of the neck portion.

The neck portion may have a cylindrical outer surface and the two toothed racks may be arranged diametrically opposite with respect to the neck portion.

Advantageously, the or each toothed rack may be connected to a collar used to attach the or each toothed rack to the neck portion of the container.

Preferably, the or each toothed rack may be formed as an integral part of the collar.

The collar may have two toothed racks formed as an integral part thereof.

The cylindrical outer surface of the neck portion may have a circumferentially extending groove formed therein in which a portion of the collar is engaged.

The circumferentially extending groove may define an annular abutment surface against which the portion of the collar reacts when the or each lever is actuated (e.g. rotated) to urge the container towards the nozzle.

The or each lever may have first and second toothed portions for engagement with respective racks attached to the container.

There may be a first lever located on one side of the container and a second lever located on an opposite side of the container each having first and second toothed portions for engagement with respective racks attached to the container, there being a first rack having a first set of teeth for engagement with the first toothed portion of the first lever and a second set of teeth for engagement with the first toothed portion of the second lever and a second rack having a first set of teeth for engagement with the second toothed portion of the first lever and a second set of teeth for engagement with the second toothed portion of the second lever.

The or each lever may be U-shaped in cross-section having first and second flanges joined together by a bridging portion.

In which case, the first flange may have an end portion forming said first toothed portion and the second flange may have an end portion forming said second toothed portion.

The or each lever may be pivotally supported within the housing by a pivotal connection between the lever and part of the housing.

The housing may have a front wall, a rear wall and two opposing side walls and the or each lever may be pivotally connected to the front and rear walls.

The housing may have a front wall, a rear wall and two opposing side walls and at least one of the front wall and the rear wall may have an aperture therein to view the level of the fluid in the container.

Each lever may project outwardly from the housing through an aperture formed in a respective one of the side walls.

The part of each lever which projects from the aperture may form a finger grip.

Alternatively, the nozzle may be formed as part of a body member and the or each lever may be pivotally supported within the housing by a pivotal connection between the lever the body member.

Suitably, a pre-load means is provided to prevent actuation of the compression pump until a pre-determined force is applied to the or each lever. The pre-load means acts such as to prevent actuation of the compression pump until a pre-determined force is applied to the finger operable means. The pre-determined force may thus, be thought of as a ‘threshold’ or ‘barrier’ force which must first be overcome before actuation of the compression pump can occur.

The quantum of pre-determined force that is to be overcome before actuation of the compression pump is enabled is selected according to various factors including characteristics of the pump, typical user profile, nature of the fluid and the desired spray characteristics.

Typically, the pre-determined force is in the range from 5 to 30N, more typically from 10 to 25N. That is to say, typically from 5 to 30N, more typically from 10 to 25N of force must be applied to the finger operable means before actuation of the compression pump is enabled. Such values tend to correspond to a force which prevents a suitable ‘barrier force’ to a weak, nondescript or unintended finger movement whilst readily being overcome by the determined finger (or thumb) action of a user. It will be appreciated that if the device is designed for use by a child or elderly patient it may have a lower pre-determined force than that designed for adult usage.

In one aspect, the pre-load means is physically interposed between the or each lever and the container.

In which case, the pre-load means may comprise of a step formed on the container which must be ridden over by the or each lever before the compression pump can be actuated wherein the step is over-ridden when the pre-determined force is applied to the or each lever.

Alternatively, the pre-load means may comprise of a step formed on the or each finger operable means (e.g. lever) which must be ridden over by the container before the compression pump can be actuated wherein the step is over-ridden when the pre-determined force is applied to the or each lever.

In yet a further alternative, the pre-load means may comprise of at least one detent formed on one of the container or the or each lever and a recess formed on the other of the container or the or each lever wherein the or each detent is able to ride out of the recess with which it is engaged when the pre-determined force is applied to the or each lever.

In another aspect the pre-load means is interposed between the housing and the container.

In which case, the pre-load means may comprise of one or more detents formed on the container for engagement with part of the housing, the or all of the detents being disengageable from the housing when the pre-determined force is applied to the or each lever so as to allow the compression pump to be actuated.

Alternatively, the pre-load means may comprise of one or more detents formed on the housing for engagement with part of the container, the or all of the detents being disengageable from the container when the pre-determined force is applied to the or each lever so as to allow the compression pump to be actuated.

In a further aspect, the pre-load means is interposed between the container and the discharge outlet.

In which case, the preload means may comprises of a step formed on the discharge outlet and at least one latching member attached to the container, the arrangement being such that, when the pre-determined force is applied to the or each lever, the or each latching member is able to ride over the step so as to allow the compression pump to be actuated.

Alternatively, the pre-load means may comprise of a recess formed on the discharge outlet and at least one latching member attached to the container, the arrangement being such that, when the pre-determined force is applied to the or each lever, the or each latching member is able to ride out of the recess so as to allow the compression pump to be actuated.

In a further aspect, the pre-load means is interposed between the housing and the or each lever.

In which case, the pre-load means may comprise of at least one detent formed on the housing for engagement with the or each lever, the or all of the detents being disengageable from the respective lever when the pre-determined force is applied to the or each lever so as to allow the compression pump to be actuated.

Alternatively, the pre-load means may comprise of at least one detent formed on the or each lever for engagement with part of the housing, the or all of the detents being disengageable from the housing when the pre-determined force is applied to the or each lever so as to allow the compression pump to be actuated.

In a further aspect, the pre-load means is interposed between the actuating means and the housing.

In which case, the pre-load means may comprise of at least one detent formed on part of the actuating means for engagement with part of the housing, the or all of the detents being disengageable from the housing when the pre-determined force is applied to the or each lever so as to allow the compression pump to be actuated.

Alternatively, the pre-load means may comprise of at least one detent formed on part of the housing each detent being arranged for engagement with a complementary recess formed on part of the actuating means, each detent being disengageable from its respective recess when the pre-determined force is applied to the or each lever so as to allow the compression pump to be actuated.

In a further aspect, the pre-load means is interposed between the or each lever and the respective actuating means.

In which case, the pre-load means may comprise of at least one detent formed on the or each lever for engagement with a respective recess formed on part of the actuating means, each detent being disengageable from its respective complementary recess when the pre-determined force is applied to the or each lever so as to allow the compression pump to be actuated.

Alternatively, the pre-load means comprises of at least one detent formed on each actuating means for engagement with a recess formed on a respective lever, each detent being disengageable from its respective complementary recess when the pre-determined force is applied to the or each lever so as to allow the compression pump to be actuated.

As yet a further alternative, the preload means may comprise of an actuating device having a variable mechanical ratio such that until the pre-determined force is applied to the or each lever no significant force is transferred to the container along the longitudinal axis.

The fluid dispensing device may alternatively comprise a single lever and the pre-load means may further comprise of a spring interposed between the lever and the container, the spring being used to urge the container towards the nozzle so as to actuate the compression pump.

In which case the spring may be compressed by movement of the lever until the pre-determined force is applied (i.e. by a combination of user-applied force and stored spring force), at which point the threshold of the pre-load means used to prevent actuation of the compression pump is overcome by the force being applied to the container such that the container moves rapidly towards the nozzle so as to actuate the compression pump.

Suitably, the fluid dispensing device is additionally provided with force modifying means for modifying the force applied to the container. That is to say, means for modifying the force applied to (and therefore, ultimately acting on) the container compared to that force directly applied to the or each lever by the user.

Suitably, the force modifying means acts such as to amplify the force applied (i.e. it comprises force amplifying means). The amplification may be provided in either a uniform manner such as by a constant amplification, for example by a ratio of from 1.5:1 to 10:1 (amplified force: initial force; i.e. degree of amplification of from 1.5 to 10), more typically from 2:1 to 5:1. In another aspect, the amplification is applied in a non-constant manner such as progressive increase or progressive decrease of mechanical advantage over the applied force cycle.

The exact profile of force modification may be readily determined by reference to the desired spray profile and all relevant characteristics of the device and formulation to be sprayed (e.g. viscosity and density).

The force modifying means may in one aspect, be integral with the or each lever. In this aspect, the force modifying means may comprise an aspect of the or each lever shaped to give rise to a mechanical advantage.

In another aspect, the force modifying means is located non-integral with the or each lever, and typically between the or each lever and the container. Again this aspect, the force modifying means may comprise an aspect of the or each lever shaped to give rise to a mechanical advantage.

In one aspect, the force modifying means only acts (i.e. only acts to modify the user applied force) once the pre-determined force has been overcome. In preferred aspects, the modifying force acts such that once the pre-determined force has been overcome the force applied to the container is either relatively constant or increases on a relatively constant basis.

In one particular aspect, the force modifying means additionally comprises a stop feature, which acts to stop force being applied to the container once either a particular maximum force is reached or more typically, once the container has been moved a particular distance. In one aspect, the stop functions to prevent excess force being applied to the compression pump.

Embodiments are envisaged in which the fluid discharge device is reversibly removable from the housing of the fluid dispensing device. In such embodiments the fluid dispensing device comprises a housing assembly and fluid discharge device receivable thereby.

According to a second aspect of the invention there is provided a fluid discharge device for use in a fluid dispensing device in accordance with the first aspect of the invention the fluid discharging device comprising a container having a longitudinal axis for storing a fluid to be dispensed and a compression pump attached to one end of the container, the pump having a suction inlet located within the container and a discharge outlet for transferring, in use, fluid from the pump to a nozzle wherein the container has at least one toothed rack attached thereto.

The or each toothed rack may extend parallel to the longitudinal axis of the container.

The or each toothed rack may be attached to the container by being formed as an integral part of a collar attached to a neck portion of the container.

Alternatively, the or each rack may be attached to the container by being formed as an integral part of the container.

According to a third aspect of the invention there is provided a housing assembly for a fluid discharge device, the housing assembly comprising a housing for moveably supporting the fluid discharge device, a body having a nozzle extending therefrom for insertion into a body cavity and at least one toothed lever pivotally supported within the housing for toothed engagement with a container forming part of the fluid discharge device.

According to a still further aspect of the present invention there is provided a kit of parts comprising a housing assembly as described above and a fluid discharge device receivable thereby. The fluid discharge device has a longitudinal axis and comprises a container for storing the fluid to be dispensed and a compression pump having a suction inlet located within the container and a discharge tube extending along the longitudinal axis for transferring fluid from the pump to the nozzle.

It is also envisaged that the housing assembly could be supplied as a separate item, into which a user or pharmacist later fits a suitable fluid discharge device.

The fluid discharge device is in one aspect in accordance with the first aspect of the invention (i.e. a compression pump-type device). In another aspect, the fluid discharge device is an aerosol container having a dispensing valve (typically, a metering valve, such as a slide valve type metering valve) of the type well-known for use in metered dose inhaler (MDI) type medicament dispensers.

Suitably, the fluid discharge device herein comprises a pre-compression pump, such as a VP3, VP7 or modifications, model manufactured by Valois SA. Typically, such pre-compression pumps are typically used with a bottle (glass or plastic) container capable of holding 8-50 ml of a formulation. Each spray will typically deliver 50-100 μl of such a formulation and the device is therefore capable of providing at least 100 metered doses.

By metered dose inhaler (MDI) it is meant a discharge device suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary.

Where the medicament container is an aerosol container, the valve typically comprises a valve body having an inlet port through which a medicament aerosol formulation may enter said valve body, an outlet port through which the aerosol may exit the valve body and an open/close mechanism by means of which flow through said outlet port is controllable.

The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 63 μl. Suitably, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined therebetween and such that during movement between is non-dispensing and dispensing positions the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation.

There may be a first lever located on one side of the container and a second lever located on an opposite side of the container.

The or each lever may have first and second toothed portions for engagement with respective racks attached to the container.

The or each lever may be U-shaped in cross-section having first and second flanges joined together by a bridging portion.

The first flange may have an end portion forming said first toothed portion and the second flange may have an end portion forming said second toothed portion.

The housing may have a front wall, a rear wall and two opposing side walls and at least one of the front wall and the rear wall may have an aperture therein to view the level of the fluid in the container.

The or each lever may be pivotally supported within the housing by a pivotal connection between the lever and part of the housing.

The housing may have a front wall, a rear wall and two opposing side walls and the or each lever may be pivotally connected to the front and rear walls.

Each lever may project outwardly from the housing through a respective aperture formed in a one of the side walls.

The part of each lever which projects from the aperture may form a finger grip.

Alternatively, the or each lever may be pivotally supported within the housing by a pivotal connection between the body and the respective lever.

The invention will now be described further with reference to the accompanying drawing in which:

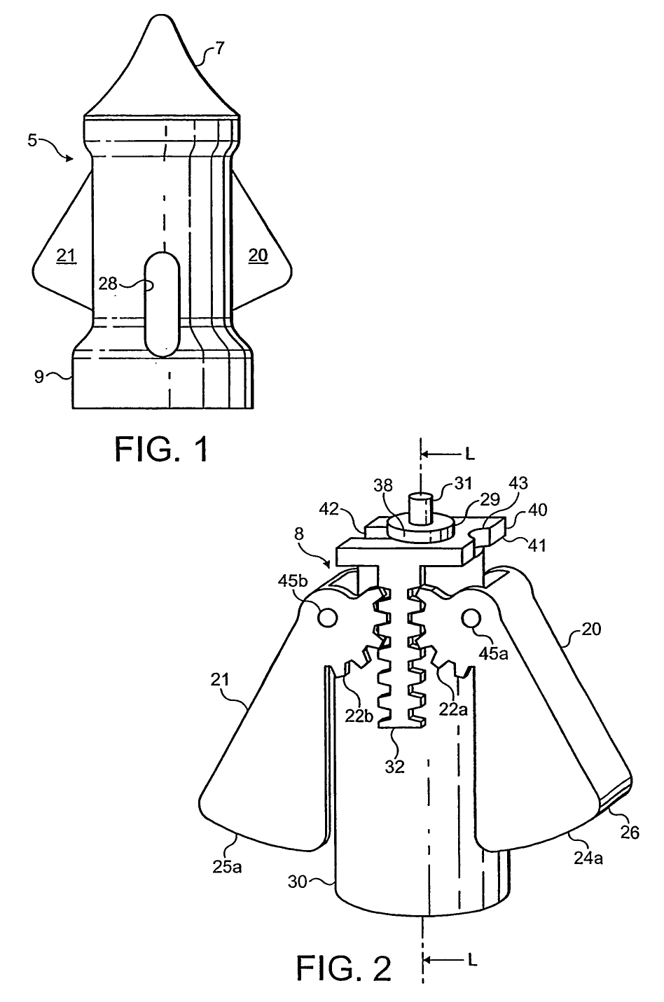

FIG. 1 is a front view a fluid dispensing device according to the invention in a ready for use state;

FIG. 2 is a pictorial view of a fluid discharge device forming part of the fluid dispensing device shown in FIG. 1;

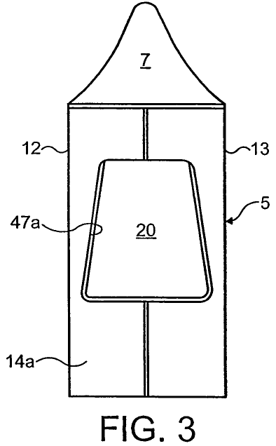

FIG. 3 is a side view of the fluid dispensing device shown in FIG. 1;

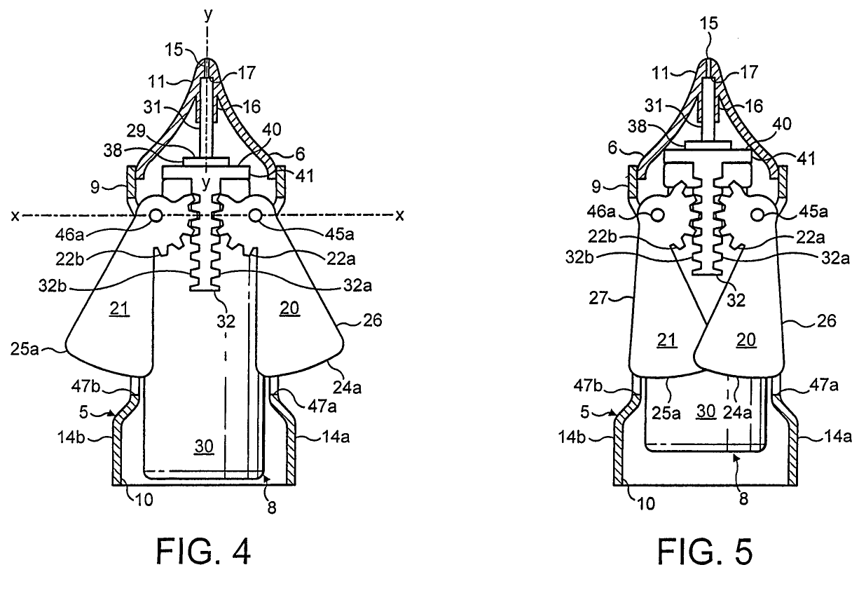

FIG. 4 is staggered cross-section through the fluid dispensing device shown in FIG. 1 showing the fluid discharge device in a ready to use state;

FIG. 5 is a staggered cross-section through the fluid dispensing device shown in FIG. 1 showing the fluid discharge device in a discharged state;

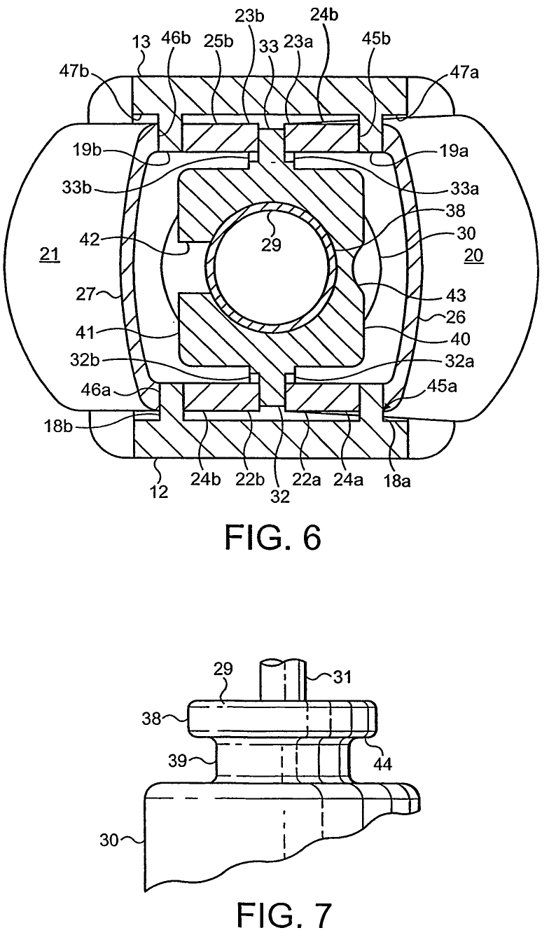

FIG. 6 is an enlarged cross-section along the line X-X on FIG. 4; and

FIG. 7 is an enlarged side view of a neck portion of fluid reservoir forming part of the fluid discharge device.

With reference to FIGS. 1 to 7 there is shown a fluid dispensing device 5 for spraying a fluid into a body cavity comprising a housing 9, a nozzle 11 for insertion into a body cavity and a fluid discharge device 8 moveably housed within the housing 9. The fluid discharge device 8 comprises of a container 30 for storing the fluid to be dispensed and a compression pump 29 having a suction inlet 32 located within the container 30 and a discharge outlet 31 at one end of the container 30 for transferring fluid from the pump to the nozzle 11. A finger operable means 20, 21 is provided to apply a force to the container 30 to move the container 30 towards the nozzle 11 so as to actuate the pump.

The finger operable means is in the form of at least one but preferably two opposing levers 20, 21 each of which is pivotally supported within the housing 9 and is driveably connected to the container 30 so as to urge the container 30 towards the nozzle 11 when each lever 20, 21 is rotated by a user. When there are two levers forming first and second levers 20, 21, then the levers 20, 21 are rotated by being squeezed together by a user.

In more detail, the fluid dispensing device 5 comprises of a housing assembly and the fluid discharge device 8. The housing assembly comprises of the housing 9 for moveably supporting the fluid discharge device 8, a body 6 having the nozzle 11 extending therefrom, a protective end cap 7 for engagement with the body 6 to protect the dispensing nozzle 11 and the two levers 20, 21 pivotally supported within the housing 9.

It is envisaged that the end cap 7 may be attached to the body by a flexible strap or tether which could be moulded as part of the end cap 7 or the end cap 7 and the body could be made as a single component.

The body 6 and the nozzle 11 are made as a single part from a plastic material such as polypropylene and the body 6 is adapted at a lower end for engagement with an upper end of the housing 9. The body 6 and the housing 9 are fixed together by any suitable means.

The housing 9 defines a cavity 10 formed by a front wall 12, a rear wall 13 and first and second end walls 14a, 14b. An aperture 28 is formed in the front wall 12 of the housing 9 so as to allow a user to view the contents of the container. If required an aperture could also be formed in the rear wall 13.

The housing 9 is formed by two separate shells which are joined together to form the housing 9. One of the shells forms the front wall 12 and part of the two side walls 14a, 14b and the other shell forms the rear wall 13 and the remaining part of the two side walls 14a, 14b.

The discharge outlet from the pump is in the form of a tubular delivery tube 31 and a tubular guide in the form of an outlet tube 16 is formed within the nozzle 11 to align and locate the delivery tube 31 correctly with respect to the nozzle 11.

An annular abutment 17 is formed at the end of the outlet tube 16. The annular abutment 17 defines the entry to an orifice 15 through which fluid can flow in use and is arranged for abutment with an end of the delivery tube 31.

The fluid discharge device 8 has a longitudinal axis L-L co-incident with a longitudinal axis of the container 30 and a longitudinal axis of the tubular delivery tube 31. The nozzle 11 has a longitudinal axis Y-Y which is aligned with the longitudinal axis L-L of the fluid discharge device 8 so that when the pump is actuated the force applied to the tubular delivery tube 31 is along the longitudinal axis of the tubular delivery tube 31 and no bending or deflection of the delivery tube 31 will occur due to the applied force.

Each of the first and second levers 20, 21 is driveably connected to the container 30 near to said one end of the container 30 where the container terminates in a neck 29.

To form the driveable connection each of the first and second lever 20,21 has a toothed portion 22a, 23a; 22b, 23b for engagement with a respective toothed rack 32, 33 attached to the container 30 and in particular to the neck portion 29 of the container 30. Each of the racks 32, 33 is arranged so as to extend parallel to the longitudinal axis of the container 30.

Each of toothed racks 32, 33 has two sets of opposed teeth, a first set of teeth 32a, 33a for engagement with the first lever 20 and a second set of teeth 32b, 33b for engagement with the second lever 21.

The neck portion 29 of the container 30 has a cylindrical outer surface 38 and the two toothed racks 32, 33 are arranged on opposite sides of the neck portion 29 so that the two toothed racks 32, 33 are arranged diametrically opposite with respect to the neck portion 29.

Each of the toothed racks 32, 33 is connected to a collar 40 used to attach the toothed racks 32, 33 to the neck portion 29 of the container 30.

The toothed racks 32, 33 are formed as an integral part of the collar 40 so that the collar 40 has the two toothed racks 32, 33 formed as an integral part of it.

The cylindrical outer surface 38 of the neck portion 29 has a circumferentially extending groove 39 formed therein in which a portion of the collar 40 in the form of a flange 41 is engaged. The flange 41 has a slot 42 formed in one edge and a depression 43 formed in an opposite edge. The slot 42 allows the flange to be expanded over the neck 29 so as to allow it to be engaged with the groove 39 and the depression 43 reduces the thickness of the flange 41 to assist in this procedure.

It will however be appreciated that other methods could be used to secure the collar 40 to the neck 29 of the container 30. However, this method is advantageous in that it allow the collar 40 to be snapped onto a standard fluid discharge device 8 without the need for special tools or equipment.

The circumferentially extending groove 39 defines an annular abutment surface 44 against which the flange portion 41 of the collar 40 can react when the two levers 20, 21 are rotated to urge the container 30 towards the nozzle 11.

The first lever 20 is located on one side of the container 30 and the second lever is located on an opposite side of the container 30. Each of the first and second levers 20, 21 has first and second toothed portions 22a, 23a; 22b, 23b for engagement with the two racks 32, 33 attached to the container 30.

A first rack 32 of the two racks has a first set of teeth 32a for engagement with the first toothed portion 22a of the first lever 20 and a second set of teeth 32b for engagement with the first toothed portion 22b of the second lever 21 and a second rack 33 of the two racks has a first set of teeth 33a for engagement with the second toothed portion 23a of the first lever 20 and a second set of teeth 33b for engagement with the second toothed portion 23b of the second lever 20.

Each of the two levers 20, 21 is U-shaped in cross-section and has first and second flanges 24a, 24b; 25a, 25b joined together by a bridging portion 26, 27.

The first flange 24a of the first lever 20 has an end portion forming said first toothed portion 22a and the second flange 24b of the first lever 20 has an end portion forming said second toothed portion 23a. Similarly, the first flange 25a of the second lever 21 has an end portion forming said first toothed portion 22b and the second flange 25b of the second lever 21 has an end portion forming said second toothed portion 23b.

Each of the levers 20, 21 is pivotally supported within the housing 9 by a pivotal connection between the respective lever 20, 21 and part of the housing 9 in the form of the front and rear walls 12 and 13.

The front wall 12 has two cylindrical pivot pins 18a, 18b formed as an integral part thereof and the rear wall 13 has two cylindrical pins 19a, 19b formed as an integral part thereof.

The first lever 20 has an aperture 45a formed in the first flange 24a for co-operation with the pivot pin 18a and an aperture 45b formed in the second flange 24b for co-operation with the pivot pin 19a. Similarly, the second lever 21 has an aperture 46a formed in the first flange 25a for co-operation with the pivot pin 18b and an aperture 46b formed in the second flange 25b for co-operation with the pivot pin 19b.

The pivot pins 18a, 19a; 18b, 19b form in combination with the apertures 45a, 45b; 46a, 46b pivotal connections at an upper end of the levers 20, 21 between the levers 20, 21 and the housing 9.

Each of the levers 20,21 projects outwardly from the housing 9 through an aperture 47a, 47b formed in a respective one of the side walls 14a, 14b.

As previously discussed the housing 9 is formed by two separate shells which are joined together to form the housing 9. Each of the apertures 47a, 47b is therefore formed by complementary cut-outs in the side walls 14a, 14b of the two shells that form the housing 9.

To assist a user in using the fluid dispensing device a part of each lever 20, 21 which projects from the aperture 47a, 47b forms a finger grip.

It will be appreciated that the levers 20, 21 could be pivotally attached to the housing in some other manner for example separate pivot pins could be used or the levers could have pivot pins formed as an integral part for co-operation with apertures formed in the housing 9. It will also be appreciated that the levers could be pivotally connected to another part of the housing such as the side walls.

It will further be appreciated that the housing could be made as one-part and that the body and the housing could be made as one part.

In addition each lever 20, 21 could be pivotally supported within the housing 9 by a pivotal connection between each lever 20, 21 and the body member 6.

The fluid discharge device 8 is in most respects conventional and will only be described briefly herein.

The fluid discharge device 8 has a longitudinal axis L-L corresponding to the longitudinal axis of the container 30. The container 30 defines a reservoir containing several doses of the fluid to be dispensed and a compression pump is attached to the neck 29 formed at one end of the container 30. The pump is used to pump the fluid out of the container 30.

The pump has a suction inlet located within the container 30 and a discharge outlet for transferring, in use, fluid from the pump to the nozzle 11.

The neck 29 of the container 30 has the two toothed racks 32, 33 attached thereto. Each of the toothed racks 32, 33 extends parallel to the longitudinal axis L-L of the container.

Preferably, each of the toothed racks 32, 33 is attached to the container by being formed as an integral part of the collar 40 attached to the neck portion 29 of the container 30.

Alternatively, each of the toothed racks can be attached to the container by being formed as an integral part of the container. That is to say if the container is moulded from plastic the two toothed racks 32, 33 are moulded as part of the container 30.

The container 30 as shown is made from a translucent or transparent plastics material however it will be appreciate that it could be made from other translucent or transparent materials such as glass in which case a collar would need to be used.

The pump includes a plunger (not shown) slidingly engaged within a pump casing which defines a chamber (not shown) sized to accommodate a single dose of fluid. The plunger is attached to the tubular delivery tube 31 which is arranged to extend from one end of the pump for co-operation with the outlet tube 16 of the dispensing nozzle 11. The plunger includes a piston (not shown) slidably supported in the chamber formed in the pump casing.

The fluid is discharged through a discharge channel defined by the tubular delivery tube 31 into the orifice 15 of the dispensing nozzle 11.

The size of chamber is such that it accommodates a single dose of fluid, the diameter of the chamber and piston combined with the stroke of the plunger being such that a full stroke of the plunger in the chamber will produce a change in volume equal to a single dose of fluid.

The pump casing is connected to the container 30 such that when the piston is moved by a return spring (not shown) into a start position a new dose of fluid is drawn into the cylinder via the suction inlet in the form of a pick-up tube from the container 30 ready for discharge.

Assembly and operation of the fluid dispensing device is as follows.

FIG. 4 shows the fluid dispensing device 5 in a ready to use state in which the two levers 20, 21 are in a ready for use position.

A fluid discharge device 8 has already been inserted into the housing 9 by lifting the two levers 20, 21 fully upward and inserting the container 30 into the housing until the delivery tube 31 fully engages with the outlet tube 16. The two levers 20, 21 have then been folded down into the position shown in FIG. 4 such that the toothed portions 22a, 23a, 22b, 23b are engaged with the racks 32, 33. The levers 20, 21 in this position are used to hold the fluid discharge device 8 within the housing 9 but if required the container 30 could be slidably engageable with one or more support structures (not shown) to assist with the location and retention of the fluid discharge device 8 in the housing 9.

The next step is for a user to grasp the fluid dispensing device 5 by the two levers 20, 21. Provided that only a light pressure is applied to the levers 20, 21 no fluid will be discharged due to static friction in the mechanism and the user is able to manoeuvre the dispensing nozzle 11 of the fluid dispensing device 5 into the body orifice into which fluid is required to be dispensed. If the user then squeezes the two levers 20, 21 together with increasing force the static friction between the toothed portions 22a, 23a, 22b, 23b and the racks 32, 33 and the levers 20, 21 and the pivot pins 18a, 19a; 18b, 19b will eventually be overcome and the interaction of the toothed portions 22a, 23a, 22b, 23b with the racks 32, 33 will then cause the container 30 to be moved rapidly towards the nozzle 11.

However, because the end of the delivery tube 31 is in abutting contact with the annular abutment 17, the delivery tube 31 cannot move in the same direction.

The effect of this is to cause the delivery tube 31 to push the plunger into the pump casing thereby moving the piston of the pump in the cylinder. This movement causes fluid to be expelled from the cylinder into the delivery tube 31. The fluid forced into the delivery tube is then transferred into the orifice 15 from where it is expelled as a fine spray into the body orifice.

At the end of the delivery stage when the fluid discharge device has been discharged the two levers 20, 21 have been rotated so that they lie close to or flush with the side walls 14a, 14b as shown in FIG. 5.

Upon releasing the pressure applied to the levers 20, 21 the delivery tube 31 is urged out of the pump casing by the internal return spring and causes fluid to be drawn up the pick-up tube 32 to re-fill the cylinder. If necessary an external spring can be provided between the nozzle 11 and the collar 40 to assist with the return action.

The actuating procedure can then be repeated until all of the fluid in the container has been used. However, only one or two doses of fluid are normally administered at a time.

When the container is empty a new fluid discharge device 8 is loaded into the housing 9 thereby restoring the fluid dispensing device 5 into a useable condition.

It is envisaged that the fluid dispensing device could be sold as two separate items. A fluid discharge device could be sold for fitment into a housing assembly and a housing assembly could be sold into which a fluid discharge device could be fitted.

Although the invention has been described in relation to an arrangement where the driving connection between the levers and the container is made via a toothed drive it will be appreciated that alternative means for providing a driving connection could be used. For example, the levers could frictionally engage with the container or another component connected to the container to move the container.

It may be appreciated that any of the parts of the dispenser device which contact the fluid may be coated with materials such as fluoropolymer materials (e.g. PTFE or FEP) which reduce the tendency of medicament to adhere thereto. Any movable parts may also have coatings applied thereto which enhance their desired movement characteristics. Frictional coatings may therefore be applied to enhance frictional contact and lubricants (e.g. silicone oil) used to reduce frictional contact as necessary.

Administration of medicament may be indicated for the treatment of mild, moderate or severe acute or chronic symptoms or for prophylactic treatment. It will be appreciated that the precise dose administered will depend on the age and condition of the patient, the particular medicament used and the frequency of administration and will ultimately be at the discretion of the attendant physician. Embodiments are envisaged in which combinations of medicaments are employed.

Appropriate medicaments may thus be selected from, for example, analgesics, e.g., codeine, dihydromorphine, ergotamine, fentanyl or morphine; anginal preparations, e.g., diltiazem; antiallergics, e.g., cromoglycate (eg as the sodium salt), ketotifen or nedocromil (eg as the sodium salt); antiinfectives e.g., cephalosporins, penicillins, streptomycin, sulphonamides, tetracyclines and pentamidine; antihistamines, e.g., methapyrilene; anti- inflammatories, e.g., beclomethasone (eg as the dipropionate ester), fluticasone (eg as the propionate ester), flunisolide, budesonide, rofleponide, mometasone (eg as the furoate ester), ciclesonide, triamcinolone (eg as the acetonide), 6α, 9α-difluoro-11β-hydroxy-16α-methyl-3-oxo-17α-propionyloxy-androsta-1,4-diene-17β-carbothioic acid S-(2-oxo-tetrahydro-furan-3-yl) ester or 6α, 9α-Difluoro-17α-[(2-furanylcarbonyl)oxy]-11β-hydroxy-16α-methyl-3-oxo-androsta-1,4-diene-17β-carbothioic acid S-fluoromethyl ester; antitussives, e.g., noscapine; bronchodilators, e.g., albuterol (eg as free base or sulphate), salmeterol (eg as xinafoate), ephedrine, adrenaline, fenoterol (eg as hydrobromide), formoterol (eg as fumarate), isoprenaline, metaproterenol, phenylephrine, phenylpropanolamine, pirbuterol (eg as acetate), reproterol (eg as hydrochloride), rimiterol, terbutaline (eg as sulphate), isoetharine, tulobuterol or 4-hydroxy-7-[2-[[2-[[3-(2-phenylethoxy)propyl]sulfonyl]ethyl]amino]ethyl-2(3H)-benzothiazolone; PDE4 inhibitors eg cilomilast or roflumilast; leukotriene antagonists eg montelukast, pranlukast and zafirlukast; [adenosine 2a agonists, eg 2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate)]*; [(α4 integrin inhibitors eg (2S)-3-[4-({[4-(aminocarbonyl)-1-piperidinyl]carbonyl}oxy)phenyl]-2-[((2S)-4-methyl-2-{[2-(2-methylphenoxy) acetyl]amino}pentanoyl)amino] propanoic acid (e.g as free acid or potassium salt)]*, diuretics, e.g., amiloride; anticholinergics, e.g., ipratropium (eg as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagons. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament and/or to minimise the solubility of the medicament in the propellant.

Preferably, the medicament is an anti-inflammatory compound for the treatment of inflammatory disorders or diseases such as asthma and rhinitis.

In one aspect, the medicament is a glucocorticoid compound, which has anti-inflammatory properties. One suitable glucocorticoid compound has the chemical name: 6α, 9α-Difluoro-17α-(1-oxopropoxy)-11β-hydroxy-16α-methyl-3-oxo-androsta-1,4-diene-17β-carbothioic acid S-fluoromethyl ester (fluticasone propionate). Another suitable glucocorticoid compound has the chemical name: 6α, 9α-difluoro-17α-[(2-furanylcarbonyl)oxy]-11β-hydroxy-16α-methyl-3-oxo-androsta-1,4-diene-17β-carbothioic acid S-fluoromethyl ester. A further suitable glucocorticoid compound has the chemical name: 6α,9α-Difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester.

Other suitable anti-inflammatory compounds include NSAIDs e.g. PDE4 inhibitors, leukotriene antagonists, iNOS inhibitors, tryptase and elastase inhibitors, beta-2 integrin antagonists and adenosine 2a agonists.

The medicament is formulated as any suitable fluid formulation, particularly a solution (e.g. aqueous) formulation or a suspension formulation, optionally containing other pharmaceutically acceptable additive components.

Suitable formulations (e.g. solution or suspension) may be stabilised (e.g. using hydrochloric acid or sodium hydroxide) by appropriate selection of pH. Typically, the pH will be adjusted to between 4.5 and 7.5, preferably between 5.0 and 7.0, especially around 6 to 6.5.

Suitable formulations (e.g. solution or suspension) may comprise one or more excipients. By the term “excipient”, herein, is meant substantially inert materials that are nontoxic and do not interact with other components of a composition in a deleterious manner including, but not limited to, pharmaceutical grades of carbohydrates, organic and inorganic salts, polymers, amino acids, phospholipids, wetting agents, emulsifiers, surfactants, poloxamers, pluronics, and ion exchange resins, and combinations thereof.

Suitable carbohydrates include monosaccharides include fructose; disaccharides, such as, but not limited to lactose, and combinations and derivatives thereof; polysaccharides, such as, but not limited to, cellulose and combinations and derivatives thereof; oligosaccharides, such as, but not limited to, dextrins, and combinations and derivatives thereof; polyols, such as but not limited to sorbitol, and combinations and derivatives thereof.

Suitable organic and inorganic salts include sodium or calcium phosphates, magnesium stearate, and combinations and derivatives thereof.

Suitable polymers include natural biodegradable protein polymers, including, but not limited to, gelatin and combinations and derivatives thereof; natural biodegradable polysaccharide polymers, including, but not limited to, chitin and starch, crosslinked starch and combinations and derivatives thereof; semisynthetic biodegradable polymers, including, but not limited to, derivatives of chitosan; and synthetic biodegradable polymers, including, but not limited to, polyethylene glycols (PEG), polylactic acid (PLA), synthetic polymers including but not limited to polyvinyl alcohol and combinations and derivatives thereof;

Suitable amino acids include non-polar amino acids, such as leucine and combinations and derivatives thereof. Suitable phospholipids include lecithins and combinations and derivatives thereof.

Suitable wetting agents, surfactants and/or emulsifiers include gum acacia, cholesterol, fatty acids including combinations and derivatives thereof. Suitable poloxamers and/or Pluronics include poloxamer 188, Pluronic® F-108, and combinations and derivations thereof. Suitable ion exchange resins include amberlite IR120 and combinations and derivatives thereof;

Suitable solution formulations may comprise a solubilising agent such as a surfactant. Suitable surfactants include α-[4-(1,1,3,3-tetramethylbutyl)phenyl]-ω-hydroxypoly(oxy-1,2-ethanediyl) polymers including those of the Triton series e.g. Triton X-100, Triton X-114 and Triton X-305 in which the X number is broadly indicative of the average number of ethoxy repeating units in the polymer (typically around 7-70, particularly around 7-30 especially around 7-10) and 4-(1,1,3,3-tetramethylbutyl)phenol polymers with formaldehyde and oxirane such as those having a relative molecular weight of 3500-5000 especially 4000-4700, particularly Tyloxapol. The surfactant is typically employed in a concentration of around 0.5-10%, preferably around 2-5% w/w based on weight of formulation.

Suitable solution formulations may also comprise hydroxyl containing organic co-solvating agents include glycols such as polyethylene glycols (eg PEG 200) and propylene glycol; sugars such as dextrose; and ethanol. Dextrose and polyethylene glycol (eg PEG 200) are preferred, particularly dextrose. Propylene glycol is preferably used in an amount of no more than 20%, especially no more than 10% and is most preferably avoided altogether. Ethanol is preferably avoided. The hydroxyl containing organic co-solvating agents are typically employed at a concentration of 0.1-20% e.g. 0.5-10%, e.g. around 1-5% w/w based on weight of formulation.

Suitable solution formulations may also comprise solublising agents such as polysorbate, glycerine, benzyl alcohol, polyoxyethylene castor oils derivatives, polyethylene glycol and polyoxyethylene alkyl ethers (e.g. Cremophors, Brij).

Suitable solution formulations may also comprise one or more of the following components: viscosity enhancing agents; preservatives; and isotonicity adjusting agents.

Suitable viscosity enhancing agents include carboxymethylcellulose, veegum, tragacanth, bentonite, hydroxypropylmethylcellulose, hydroxypropylcellulose, hydroxyethylcellulose, poloxamers (eg. poloxamer 407), polyethylene glycols, alginates xanthym gums, carageenans and carbopols.

Suitable preservatives include quaternary ammonium compounds (e.g. benzalkonium chloride, benzethonium chloride, cetrimide and cetylpyridinium chloride), mercurial agents (e.g. phenylmercuric nitrate, phenylmercuric acetate and thimerosal), alcoholic agents (e.g. chlorobutanol, phenylethyl alcohol and benzyl alcohol), antibacterial esters (e.g. esters of para-hydroxybenzoic acid), chelating agents such as disodium edetate (EDTA) and other anti-microbial agents such as chlorhexidine, chlorocresol, sorbic acid and its salts and polymyxin.

Suitable isotonicity adjusting agents act such as to achieve isotonicity with body fluids (e.g. fluids of the nasal cavity), resulting in reduced levels of irritancy associated with many nasal formulations. Examples of suitable isotonicity adjusting agents are sodium chloride, dextrose and calcium chloride.

Suitable suspension formulations comprise an aqueous suspension of particulate medicament and optionally suspending agents, preservatives, wetting agents or isotonicity adjusting agents.

The particulate medicament suitably has a mass mean diameter (MMD) of less than 20 μm, preferably between 0.5-10 μm, especially between 1-5 μm. If particle size reduction is necessary, this may be achieved by techniques such as micronisation and/or microfluidisation.

Suitable suspending agents include carboxymethylcellulose, veegum, tragacanth, bentonite, methylcellulose and polyethylene glycols.

Suitable wetting agents function to wet the particles of medicament to facilitate dispersion thereof in the aqueous phase of the composition. Examples of wetting agents that can be used are fatty alcohols, esters and ethers. Preferably, the wetting agent is a hydrophilic, non-ionic surfactant, most preferably polyoxyethylene (20) sorbitan monooleate (supplied as the branded product Polysorbate 80).

Suitable preservatives and isotonicity adjusting agents are as described above in relation to solution formulations.

The dispensing device herein is suitable for dispensing fluid medicament formulations for the treatment of inflammatory and/or allergic conditions of the nasal passages such as rhinitis e.g. seasonal and perennial rhinitis as well as other local inflammatory conditions such as asthma, COPD and dermatitis.

A suitable dosing regime would be for the patient to inhale slowly through the nose subsequent to the nasal cavity being cleared. During inhalation the formulation would be applied to one nostril while the other is manually compressed. This procedure would then be repeated for the other nostril. Typically, one or two inhalations per nostril would be administered by the above procedure up to three times each day, ideally once daily. Each dose, for example, may deliver 5 μg, 50 μg, 100 μg, 200 μg or 250 μg of active medicament. The precise dosage is either known or readily ascertainable by those skilled in the art.

The application of which this description and claims form part may be used as a basis for priority in respect of any subsequent application. The claims of such subsequent application may be directed to any feature or combination of features described therein. They may take the form of product, method or use claims and may include, by way of example and without limitation, one or more of the following claims.

1. A fluid dispensing device for spraying a fluid into a body cavity comprising:
a. a housing,
b. a nozzle for insertion into a body cavity,
c. a fluid discharge device moveably housed within the housing, the fluid discharge device comprising
1.) a container for storing the fluid to be dispensed and
2.) a compression pump having
i.) a suction inlet located within the container and
ii.) a discharge outlet at one end of the container for transferring fluid from the pump to the nozzle and
iii). a finger operable means to apply a force to the container to move the container towards the nozzle so as to actuate the pump
wherein the finger operable means comprises of at least one lever pivotally supported within the housing and driveably connected to the container so as to urge the container towards the nozzle when the or each lever is actuated by a user.

2. The fluid dispensing device of claim 1 in which there are two opposing levers each of which is pivotally connected to part of the housing and is driveably connected to the container so as to urge the container towards the nozzle when the levers are squeezed together by a user.

3. The fluid dispensing device of claim 1 in which the or each lever is driveably connected to the container near to said one end of the container.

4. The fluid dispensing device of claim 1 in which the or each lever has a toothed portion for engagement with a toothed rack attached to the container so as to form the driveable connection therebetween.

5. The fluid dispensing device of claim 4 in which the container has a longitudinal axis and the or each toothed rack extends parallel to the longitudinal axis of the container.

6. The fluid dispensing device of claim 4 in which each toothed rack has two sets of opposed teeth, a first set of teeth for engagement with a first lever and a second set of teeth for engagement with a second lever.

7. The fluid dispensing device of claim 4, in which the container has a neck portion at said one end and the or each toothed rack is attached to the neck portion of the container so as to form in combination with each lever the driveable connection.

8. The fluid dispensing device of claim 4, in which the container has two toothed racks attached thereto.

9. The fluid dispensing device of claim 7 in which the container has two toothed racks attached thereto and in which the two toothed racks are arranged on opposite sides of the neck portion.

10. The fluid dispensing device of claim 9, in which the neck portion has a cylindrical outer surface and the two toothed racks are arranged diametrically opposite with respect to the neck portion.

11. The fluid dispensing device of claim 7, in which the or each toothed rack is connected to a collar used to attach the or each toothed rack to the neck portion of the container.

12. The fluid dispensing device of claim 11, in which the or each toothed rack formed as an integral part of the collar.

13. The fluid dispensing device of claim 12, in which the collar has two toothed racks formed as an integral part thereof.

14. The fluid dispensing device of claim 11, in which the cylindrical outer surface of the neck portion has a circumferentially extending groove formed therein in which a portion of the collar is engaged.

15. The fluid dispensing device of claim 14, in which the circumferentially extending groove defines an annular abutment surface against which the portion of the collar reacts when the or each lever is rotated to urge the container towards the nozzle.

16. The fluid dispensing device of claim 4, in which the or each lever has first and second toothed portions for engagement with respective racks attached to the container.

17. The fluid dispensing device of claim 16, in which there is a first lever located on one side of the container and a second lever located on an opposite side of the container each having first and second toothed portions for engagement with respective racks attached to the container, therebeing a first rack having a first set of teeth for engagement with the first toothed portion of the first lever and a second set of teeth for engagement with the first toothed portion of the second lever and a second rack having a first set of teeth for engagement with the second toothed portion of the first lever and a second set of teeth for engagement with the second toothed portion of the second lever.

18. The fluid dispensing device of claim 16, in which the or each lever is U-shaped in cross-section having first and second flanges joined together by a bridging portion.

19. The fluid dispensing device of claim 18, in which the first flange has an end portion forming said first toothed portion and the second flange has an end portion forming said second toothed portion.

20. The fluid dispensing device of claim 1, in which the or each lever is pivotally supported within the housing by a pivotal connection between the lever and a part of the housing.

21. The fluid dispensing device of claim 20, in which the housing has a front wall, a rear wall and two opposing side walls and the or each lever is pivotally connected to the front and rear walls.

22. The fluid dispensing device of claim 20, in which the housing has a front wall, a rear wall and two opposing side walls and at least one of the front wall and the rear wall has an aperture therein to view the level of the fluid in the container.

23. The fluid dispensing device of claim 21, in which each lever projects outwardly from the housing through an aperture formed in a respective one of the side walls.

24. The fluid dispensing device of claim 23, in which the part of each lever which projects from the aperture forms a finger grip.

25. The fluid dispensing device of claim 4, in which the nozzle is formed as a part of a body member and the or each lever is pivotally supported within the housing by a pivotal connection between the lever and the body member.

26. The fluid dispensing device of claim 1, wherein the container contains a volume of fluid medicament formulation.

27. The fluid dispensing device of claim 26, wherein said fluid medicament formulation is in the form of a solution formulation.

28. The fluid dispensing device of claim 26, wherein said fluid medicament formulation is in the form of a suspension formulation.

29. The fluid dispensing device of claim 26, wherein the fluid medicament formulation comprises an anti-inflammatory medicament compound.

30. The fluid dispensing device of claim 29, wherein said medicament compound is a glucocorticoid compound.

31. The fluid dispensing device of claim 30, wherein said glucocorticoid compound is selected from the group consisting of 6α, 9α-Difluoro-17α-(1-oxopropoxy)-11β-hydroxy-16α-methyl-3-oxo-androsta-1,4-diene-17β-carbothioic acid S-fluoromethyl ester; 6α, 9α-difluoro-17α-[(2-furanylcarbonyl)oxy]-11β-hydroxy-16α-methyl-3-oxo-androsta-1,4-diene-17β-carbothioic acid S-fluoromethyl ester; and 6α,9α-Difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester.

32. The fluid dispensing device of claim 29, wherein said medicament compound is selected from the group consisting of PDE4 inhibitors, leukotriene antagonists, iNOS inhibitors, tryptase and elastase inhibitors, beta-2 integrin antagonists and adenosine 2a agonists.

33. A fluid discharge device for use in the fluid dispensing device of claim 1 wherein the fluid discharging device comprises of a container having a longitudinal axis for storing a fluid to be dispensed and a compression pump attached to one end of the container, the pump having a suction inlet located within the container and a discharge outlet for transferring, in use, fluid from the pump to a nozzle wherein the container has at least one toothed rack attached thereto.

34. The fluid discharge device of claim 33 in which the or each toothed rack extends parallel to the longitudinal axis of the container.

35. The fluid discharge device of claim 33 in which the or each toothed rack is attached to the container by being formed as an integral part of a collar attached to a neck portion of the container.

36. The fluid dispensing device of claim 33, in which the or each rack is attached to the container by being formed as an integral part of the container.

37. A housing assembly for a fluid discharge device, the housing assembly comprising a housing for moveably supporting the fluid discharge device, a body having a nozzle extending therefrom for insertion into a body cavity and at least one toothed lever pivotally supported within the housing for toothed engagement with a container forming part of the fluid discharge device.

38. The hosing assembly of claim 37 in which there is a first lever located on one side of the container and a second lever located on an opposite side of the container.

39. The hosing assembly of claim 37 in which the or each lever has first and second toothed portions for engagement with respective racks attached to the container.

40. The hosing assembly of claim 39 in which the or each lever is U-shaped in cross-section having first and second flanges joined together by a bridging portion.

41. The hosing assembly of claim 40 in which the first flange has an end portion forming said first toothed portion and the second flange has an end portion forming said second toothed portion.

42. The hosing assembly of claim 37 in which the housing has a front wall, a rear wall and two opposing side walls and at least one of the front wall and the rear wall has an aperture therein to view the level of the fluid in the container.

43. The hosing assembly of claim 37 in which the or each lever is pivotally supported within the housing by a pivotal connection between the lever and part of the housing.

44. The hosing assembly of claim 43 in which the housing has a front wall, a rear wall and two opposing side walls and the or each lever is pivotally connected to the front and rear walls.

45. The hosing assembly of claim 42 in which each lever projects outwardly from the housing through a respective aperture formed in a one of the side walls.

46. The hosing assembly of claim 45 in which the part of each lever which projects from the aperture forms a finger grip.

47. The hosing assembly of claim 37 in which the or each lever is pivotally supported within the housing by a pivotal connection between the body and the respective lever.

48-50. (canceled)
